(19) United States
(12) Patent Application Publication
(10) Pub. No.: US 2011/0112424 A1
(43) Pub. Date: May 12, 2011

(54) DETERMINING FUNCTIONAL RESIDUAL LUNG CAPACITY

(76) Inventors: Yitzhak Kesselman, Lod (IL); Mark Shahar, Tel-Aviv (IL)

(21) Appl. No.: 12/988,931

(22) PCT Filed: Apr. 23, 2009
(86) PCT No.: PCT/IB09/51677

§ 371 Date: Dec. 15, 2010

Related U.S. Application Data
(60) Provisional application No. 61/047,114, filed on Apr. 23, 2008.

(51) Int. Cl. A61B 5/091; A61B 5/08; A61M 16/00

(52) U.S. Cl. 600/532; 600/538; 128/204.23

(57) ABSTRACT

Determining functional residual lung capacity (FRC) by changing a subject's inspirium FiO₂ by a predetermined amount, and a) for each breath in a series of breaths subsequent to changing the FiO₂, determining expiratory tidal volume, determining expiratory fractional N₂ tidal volume, multiplying the expiratory tidal volume by an absolute difference between the expiratory fractional N₂ tidal volume of the breath and that of an immediately preceding breath for a first multiplication result, dividing the first multiplication result by the sum of the differences for a first division result, and multiplying the fractional N₂ tidal volume by the sum of the first division results of the breaths for a second multiplication result, and b) dividing the sum of the second multiplication results of the breaths by the absolute difference between the fractional N₂ tidal volume of the first and last breaths to produce a measurement of the subject's FRC.

### FIELD OF THE INVENTION

The present invention relates to medical devices and methods in general, and more particularly to systems and methods for determining functional residual lung capacity of subjects.

### BACKGROUND OF THE INVENTION

The functional residual lung capacity (FRC) of a human or animal subject refers to the amount of air present in the subject's lungs at the end of passive expiration. In medicine, for example, FRC is a critical measurement which indicates whether there is enough lung tissue available to participate in the gas exchange process. This is true for non-ventilated patients with chronic lung diseases, as well as for patients requiring mechanical ventilation to assist or replace spontaneous breathing. While a patient is on a ventilator, FRC measurements are required in order to assess the condition of the patient's lungs and respiratory system, and knowledge of a patient's FRC is vital for diagnosis and treatment. Unfortunately, current methods for measuring FRC often require placing a subject in a plethysmograph, which is not feasible for a patient on a ventilator. Accordingly, FRC is often difficult to determine and monitor.

Accurate and easy-to-use systems and methods for determining FRC would therefore be advantageous.

### SUMMARY OF THE INVENTION

The present invention in embodiments thereof discloses novel systems and methods for determining functional residual lung capacity of subjects.

In one aspect of the invention a method is provided for determining the functional residual lung capacity of a subject, the method including changing the FiO₂ of a subject's inspirium by a predetermined amount, a) for each breath in a series of breaths of the subject subsequent to changing the FiO₂, determining an expiratory tidal volume measurement value of the breath, determining an expiratory fractional N₂ tidal volume measurement value of the breath, multiplying the expiratory tidal volume measurement value of the breath by an absolute difference between the expiratory fractional N₂ tidal volume measurement value of the breath and that of a breath immediately preceding the breath, thereby yielding a first multiplication result, dividing the first multiplication result by the sum of the absolute differences of each of the breaths, thereby yielding a first division result, and multiplying the expiratory fractional N₂ tidal volume measurement value of the breath by the sum of the first division results of each of the breaths, thereby yielding a second multiplication result, and b) dividing the sum of the second multiplication results of each of the breaths by the absolute difference between the expiratory fractional N₂ tidal volume measurement values of the first and last breaths in the series of breaths, thereby producing a functional residual lung capacity measurement of the subject.

In another aspect of the invention the changing step includes increasing the FiO₂.

In another aspect of the invention the changing step includes decreasing the FiO₂.

In another aspect of the invention the changing step includes changing the FiO₂ by an amount that is within the range of about 20% to about 25% of total inspired volume of the subject.

In another aspect of the invention the changing step includes changing the FiO₂ in accordance with a single step function.

In another aspect of the invention the method further includes determining a fractional expiratory CO₂ tidal volume of expirium of the subject and determining a fractional expiratory O₂ tidal volume of expirium of the subject, where the step of determining the fractional expiratory N₂ tidal volume includes determining the fractional expiratory N₂ tidal volume as a function of the O₂ and CO₂ fractional expiratory O₂ tidal volumes.

In another aspect of the invention the step of determining the fractional expiratory CO₂ tidal volume includes determining prior to changing the FiO₂.

In another aspect of the invention the step of determining the fractional expiratory CO₂ tidal volume includes determining the fractional expiratory CO₂ tidal volume separately for each of the breath in the series of breaths.

In another aspect of the invention the determining steps include determining until any of the expiratory fractional tidal volumes reaches a steady state.

In another aspect of the invention the determining steps include determining until consecutive ones of any of the expiratory fractional tidal volumes differ by less than a predefined amount.

In another aspect of the invention the determining steps include determining until consecutive ones of any of the expiratory fractional tidal volumes differ by less than <1%.

In another aspect of the invention the determining steps include determining for predefined number of breaths after any of the expiratory fractional tidal volumes reaches a steady state.

In another aspect of the invention the step of determining the fractional expiratory O₂ tidal volume includes determining using a minimal level of O₂ in the breath after the FiO₂ is increased.

In another aspect of the invention the step of determining the fractional expiratory O₂ tidal volume includes determining using a maximal level of O₂ in the breath after the FiO₂ is decreased.

In another aspect of the invention the determining steps include associating any of the tidal volumes with any of the breaths where the measurement of the tidal volume is closest in time to the occurrence of the breath after a change in detected in inspirium FiO₂ of the subject.

In another aspect of the invention a functional residual lung capacity measurement system is provided, the system including a ventilation system and a functional residual capacity analyzer configured to co-operate with the ventilation system to determine the functional residual lung capacity of a subject that is insufflated with O₂ by the ventilation system, where the analyzer is configured to a) for each breath in a series of breaths of the subject subsequent to the occurrence of a change in the FiO₂ of a subject's inspirium by a predetermined amount, determine an expiratory tidal volume measurement value of the breath, determine an expiratory fractional N₂ tidal volume measurement value of the breath, multiply the expiratory tidal volume measurement value of the breath by an absolute difference between the expiratory fractional N₂ tidal volume measurement value of the breath and that of a breath immediately preceding the breath, thereby yielding a first multiplication result, divide the first multiplication result by the sum of the absolute differences of each of the breaths, thereby yielding a first division result, and multiply the expiratory fractional N₂ tidal volume measurement value of the breath by the sum of the first division results of each of the breaths, thereby yielding a second multiplication result, and b) divide the sum of the second multiplication results of each of the breaths by the absolute difference between the expiratory fractional N₂ tidal volume measurement values of the first and last breaths in the series of breaths, thereby producing a functional residual lung capacity measurement of the subject.

In another aspect of the invention the ventilation system includes an O₂ source, an O₂ sensor configured to measure inspiratory O₂ between the O₂ source and a subject, and a flow transducer configured to measure pressure along expiratory and inspiratory channels intermediate the O₂ source and the subject, where the functional residual capacity analyzer is configured to determine any of the tidal volumes using any of the pressure measurement and the inspiratory O₂ measurement.

In another aspect of the invention the analyzer is configured to automatically initiate a measurement of the functional residual lung capacity after the change in the FiO₂ occurs.

In another aspect of the invention the analyzer is configured to cause the O₂ source to change the FiO₂ of the subject inspirium by the predetermined amount.

In another aspect of the invention the O₂ source is configured to change the FiO₂ of the subject inspirium by increasing the FiO₂.

In another aspect of the invention the O₂ source is configured to change the FiO₂ of the subject inspirium by decreasing the FiO₂.

In another aspect of the invention the O₂ source is configured to change the FiO₂ by an amount that is within the range of about 20% to about 25% of total inspired volume of the subject.

In another aspect of the invention the O₂ source is configured to change the FiO₂ in accordance with a single step function.

In another aspect of the invention the analyzer is configured to determine a fractional expiratory CO₂ tidal volume of expirium of the subject, determine a fractional expiratory O₂ tidal volume of expirium of the subject, and determine the fractional expiratory N₂ tidal volume as a function of the O₂ and CO₂ fractional expiratory O₂ tidal volumes.

In another aspect of the invention the analyzer is configured to determine the fractional expiratory CO₂ tidal volume prior to said change in FiO₂.

In another aspect of the invention the analyzer is configured to determine the fractional expiratory CO₂ tidal volume separately for each of the breath in the series of breaths.

In another aspect of the invention the analyzer is configured to make any of the determinations until any of the expiratory fractional tidal volumes reaches a steady state.

In another aspect of the invention the analyzer is configured to make any of the determinations until consecutive ones of any of the expiratory fractional tidal volumes differ by less than a predefined amount.

In another aspect of the invention the analyzer is configured to make any of the determinations until consecutive ones of any of the expiratory fractional tidal volumes differ by less than <1%.

In another aspect of the invention the analyzer is configured to make any of the determinations for predefined number of breaths after any of the expiratory fractional tidal volumes reaches a steady state.

In another aspect of the invention the analyzer is configured to determine the fractional expiratory O₂ tidal volume using a minimal level of O₂ in the breath after the FiO₂ is increased.

In another aspect of the invention the analyzer is configured to determine the fractional expiratory O₂ tidal volume using a maximal level of O₂ in the breath after the FiO₂ is decreased.

In another aspect of the invention the analyzer is configured to associate any of the tidal volumes with any of the breaths where the measurement of the tidal volume is closest in time to the occurrence of the breath after a change in detected in inspirium FiO₂ of the subject.

In another aspect of the invention a computer program product is provided for determining the functional residual lung capacity of a subject, the computer program product including a computer readable medium and computer program instructions operative to a) for each breath in a series of breaths of the subject subsequent to the occurrence of a change in the FiO₂ of a subject's inspirium by a predetermined amount, determine an expiratory tidal volume measurement value of the breath, determine an expiratory fractional N₂ tidal volume measurement value of the breath, multiply the expiratory tidal volume measurement value of the breath by an absolute difference between the expiratory fractional N₂ tidal volume measurement value of the breath and that of a breath immediately preceding the breath, thereby yielding a first multiplication result, divide the first multiplication result by the sum of the absolute differences of each of the breaths, thereby yielding a first division result, and multiply the expiratory fractional N₂ tidal volume measurement value of the breath by the sum of the first division results of each of the breaths, thereby yielding a second multiplication result, and b) divide the sum of the second multiplication results of each of the breaths by the absolute difference between the expiratory fractional N₂ tidal volume measurement values of the first and last breaths in the series of breaths, thereby producing a functional residual lung capacity measurement of the subject, where the program instructions are stored on the computer readable medium.

### BRIEF DESCRIPTION OF THE DRAWINGS

The invention will be understood and appreciated more fully from the following detailed description taken in conjunction with the appended drawings in which:

FIG. 1 is a simplified block diagram of a system for determining functional residual lung capacity of subjects, constructed and operative in accordance with an embodiment of the invention;

FIGS. 2A and 2B, taken together, is a simplified flowchart illustration of an exemplary method for determining functional residual lung capacity of subjects, operative in accordance with an embodiment of the invention;

FIG. 3 is an exemplary set of measurements useful in understanding the method of FIGS. 2A and 2B; and

FIG. 4 is a simplified block diagram of an exemplary hardware implementation of a computing system in accordance an embodiment of the invention.

### DETAILED DESCRIPTION OF THE INVENTION

The invention is now described within the context of one or more embodiments, although the description is intended to be illustrative of the invention as a whole, and is not to be construed as limiting the invention to the embodiments shown. It is appreciated that various modifications may occur to those skilled in the art that, while not specifically shown herein, are nevertheless within the true spirit and scope of the invention.

As will be appreciated by one skilled in the art, the invention may be embodied as a system, method or computer program product. Accordingly, the invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the invention may take the form of a computer program product embodied in any tangible medium of expression having computer usable program code embodied in the medium.

Any combination of one or more computer usable or computer readable medium(s) may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CDROM), an optical storage device, a transmission media such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The invention is described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowchart and/or block diagram block or blocks.

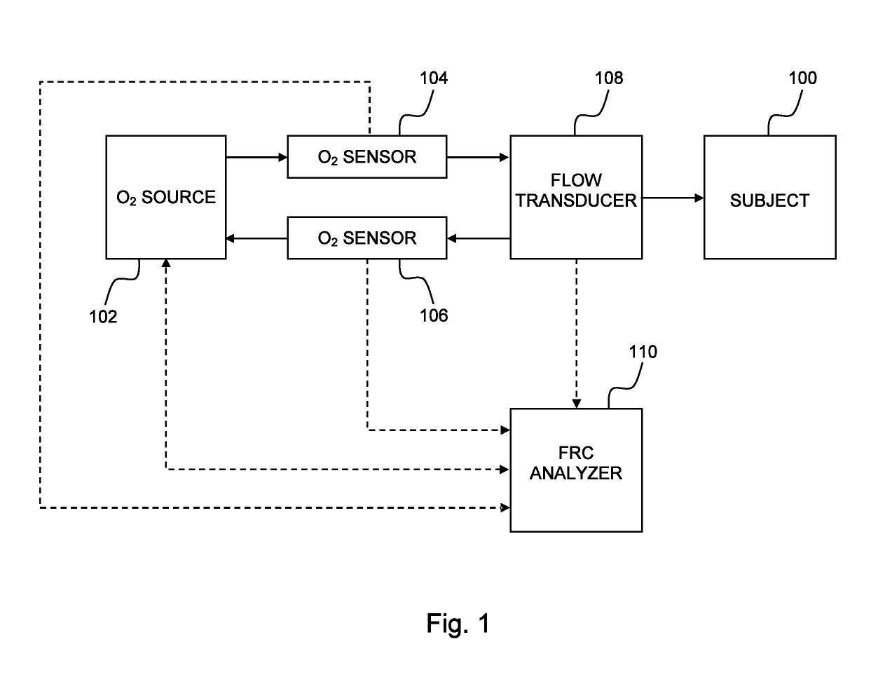

Reference is now made to FIG. 1 which is a simplified block diagram of a system for determining functional residual lung capacity of subjects, constructed and operative in accordance with an embodiment of the invention. In the system of FIG. 1, a ventilation system is shown in which a human or animal subject 100 is connected to an O₂ source 102, such as to a mechanical ventilator, using conventional techniques. An O₂ sensor 104 measures inspiratory O₂ between O₂ source 102 and subject 100 along an inspiratory channel, such as an inspirium tube, while an O₂ sensor 106 measures expiratory O₂ between O₂ source 102 and subject 100 along an expiratory channel, such as an expirium tube. A flow transducer 108 measures differential, dynamic, and static pressure signals along the expiratory and inspiratory channels, such as may be used to calculate the volumetric flow and tidal volume of subject 100. End tidal CO₂ concentration of the expirium of subject 100 may also be measured using any known means, such as by O₂ source 102 or flow transducer 108. A functional residual capacity analyzer 110 receives the various measurements described above from O₂ sensors 104 and 106 and transducer 108, as well as information from O₂ source 102, such as differential pressure signals or other acquired signals resulting from the flow of air in and out of subject 100's respiratory system, and calculates the functional residual lung capacity of subject 100 using this information as described in greater detail hereinbelow.

In one embodiment, functional residual capacity analyzer 110 is configured to initiate a measurement of the functional residual lung capacity of subject 100 by causing O₂ source 102 to increase or decrease the FiO₂ of subject 100's inspirium by a predetermined amount, such as within the range of about 20% to about 25% of total inspired volume, preferably in accordance with a single predefined step function. Additionally or alternatively, analyzer 110 is configured to automatically initiate a measurement of the functional residual lung capacity of subject 100 after such an predefined increase or decrease in the FiO₂ of subject 100's inspirium occurs, such as may be detected by any of the elements of FIG. 1 described herein.

Although two O₂ sensors 104 and 106 are shown for measuring O₂ along separate inspiratory and expiratory channels, it will be appreciated that a single O₂ sensor may alternatively be used along a single inspiratory/expiratory channel for measuring both inspiratory and expiratory O₂, provided that mutually exclusive measurement of inspiratory and expiratory gasses can be ensured.

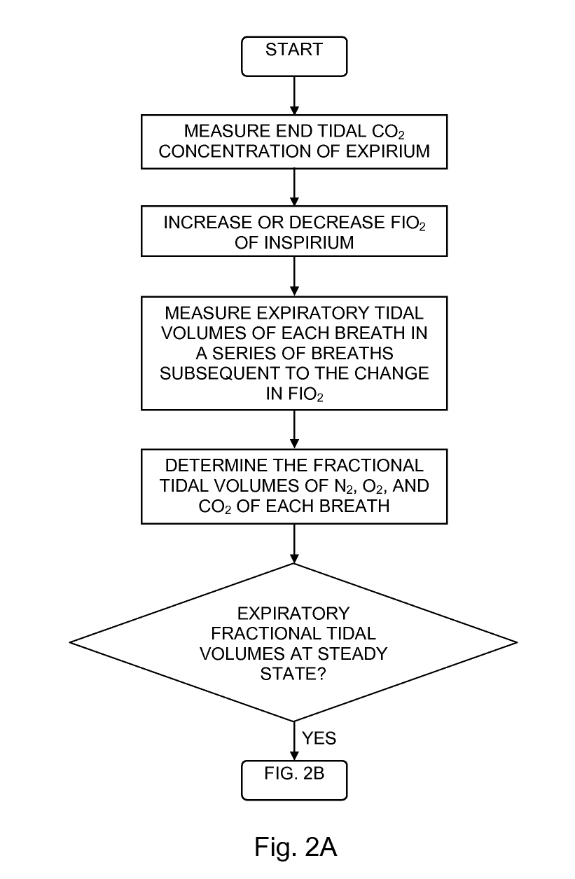

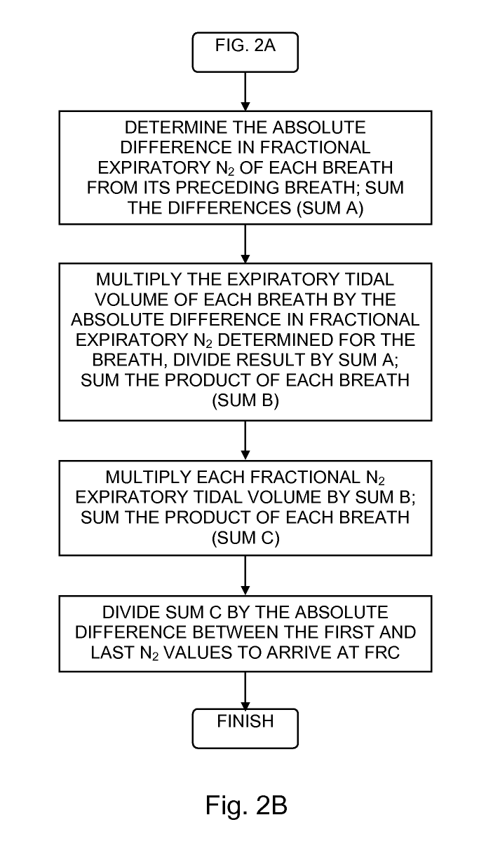

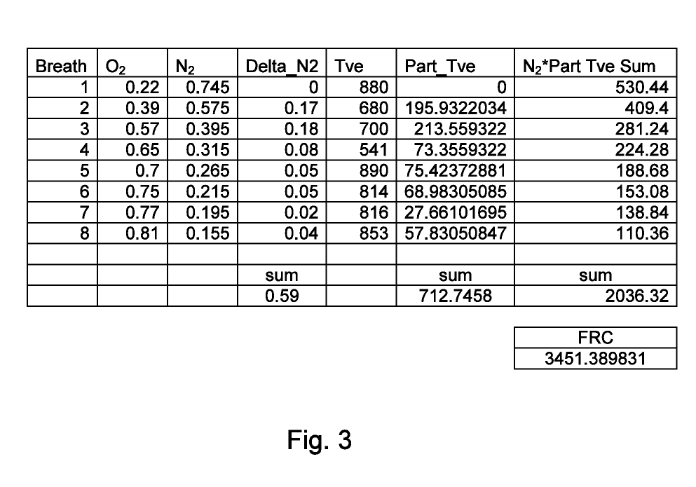

Reference is now made to FIGS. 2A and 2B, which, taken together, is a simplified flowchart illustration of a method for determining the functional residual lung capacity of a subject, operative in accordance with an embodiment of the invention. In the method of FIGS. 2A and 2B, which may be implemented using the system of FIG. 1 or any other suitable arrangement capable of providing the measurements described herein, the end tidal CO₂ concentration of the expirium of a subject is preferably determined using conventional techniques at the beginning of a series of breaths or separately for each breath. The FiO₂ of the subject's inspirium is then increased or decreased by a predetermined amount, such as within the range of about 20% to about 25% of the subject's total inspired volume, preferably in accordance with a single step function. For each breath in a series of the subject's breaths subsequent to the change in FiO₂, the expiratory tidal volume and fractional expiratory N₂ tidal volume of the breath are determined using conventional techniques, with the fractional expiratory N₂ tidal volume preferably being determined as a function of measured expiratory O₂ and CO₂. In one embodiment, the previously-determined end tidal CO₂ concentration is assumed to be constant for each breath if the end tidal CO₂ concentration is not measured for each breath. The expiratory and fractional tidal volumes are preferably determined for each breath in the series of breaths until the expiratory fractional tidal volumes reach steady state, such as where consecutive N₂ or O₂ fractional tidal volumes differ by less than a predefined amount, such as <1%. Optionally, the expired and fractional tidal volumes may also be determined for a predefined number of post-steady state breaths. An exemplary set of such measurements is shown in a table in FIG. 3, to which additional reference is now made, the table having columns for expiratory N₂ and O₂ fractional tidal volumes expressed as fractions of total expiratory tidal volume, as well as for the expiratory tidal volume of each breath in the series in the column labeled “Tve”.

An absolute difference in expiratory N₂, shown in the column labeled “Delta N₂,” is determined for each breath in the series of breaths as the difference between the fractional expiratory N₂ tidal volume of the breath and that of the breath immediately preceding it, where an absolute difference of zero may be used for the first breath in the series. The expiratory tidal volume of each breath is then multiplied by the absolute difference in expiratory N₂ determined for the breath, and the result is divided by the sum of the N₂ absolute differences for each of the breaths in the series, with the results shown in the column labeled “Part Tve,” which results are summed. Each fractional N₂ expiratory tidal volume is then multiplied by the sum of the Part Tve values, with the results shown in the column labeled “N₂*Part Tve Sum,” which results are summed. The sum of the N₂*Part Tve values is then divided by the absolute difference between the first and last fractional N₂ values to arrive at a functional residual capacity value expressed in cubic centimeters.

If the series of breaths are measured as above during an FiO₂ increase, the minimal level of O₂ in each breath is preferably measured, whereas if the measurements are performed during an FiO₂ decrease, the maximal level of O₂ in each breath is preferably measured.

Synchronization between breaths and measurements is preferably achieved as follows. Once an increase or decrease is detected in inspirium FiO2 for a given breath, the acquired tidal volume closest in time subsequent to the increase or decrease detection is related to this breath. Thereafter, although there is typically a delay in measuring expired O₂, the subject's next breaths are assumed to be affected by the change in FiO₂.

Although gas and tidal volume measurements of inspirium are not directly relied upon for determining a subject's functional residual lung capacity, such measurements are preferably used for synchronization between breaths, detecting system leaks and other anomalies such as equipment malfunction, determining the accuracy of the sensing equipment, and determining the amount of O₂ consumed during each breath.

It will be appreciated that any of the elements described hereinabove may be implemented as a computer program product embodied in a computer-readable medium, such as in the form of computer program instructions stored on magnetic or optical storage media or embedded within computer hardware, and may be executed by or otherwise accessible to a computer.

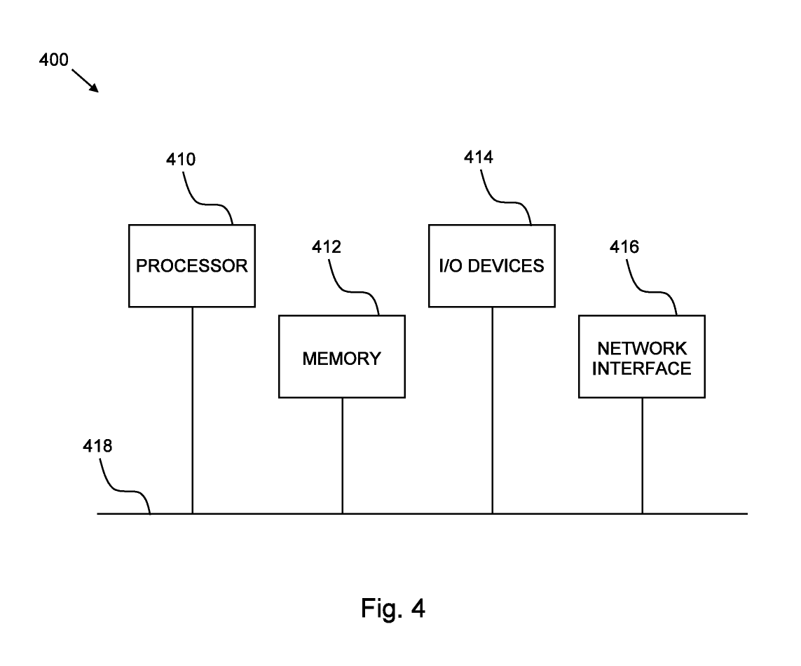

Referring now to FIG. 4, block diagram 400 illustrates an exemplary hardware implementation of a computing system in accordance with which one or more components/methodologies of the invention (e.g., components/methodologies described in the context of FIGS. 1-3) may be implemented, according to an embodiment of the invention.

As shown, the techniques for controlling access to at least one resource may be implemented in accordance with a processor 410, a memory 412, I/O devices 414, and a network interface 416, coupled via a computer bus 418 or alternate connection arrangement.

It is to be appreciated that the term “processor” as used herein is intended to include any processing device, such as, for example, one that includes a CPU (central processing unit) and/or other processing circuitry. It is also to be understood that the term “processor” may refer to more than one processing device and that various elements associated with a processing device may be shared by other processing devices.

The term “memory” as used herein is intended to include memory associated with a processor or CPU, such as, for example, RAM, ROM, a fixed memory device (e.g., hard drive), a removable memory device (e.g., diskette), flash memory, etc. Such memory may be considered a computer readable storage medium.

In addition, the phrase “input/output devices” or “I/O devices” as used herein is intended to include, for example, one or more input devices (e.g., keyboard, mouse, scanner, etc.) for entering data to the processing unit, and/or one or more output devices (e.g., speaker, display, printer, etc.) for presenting results associated with the processing unit.

The flowchart and block diagrams in the Figures illustrate the architecture, functionality, and operation of possible implementations of systems, methods and computer program products according to various embodiments of the invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the methods and apparatus herein may or may not have been described with reference to specific computer hardware or software, it is appreciated that the methods and apparatus described herein may be readily implemented in computer hardware or software using conventional techniques.

While the invention has been described with reference to one or more specific embodiments, the description is intended to be illustrative of the invention as a whole and is not to be construed as limiting the invention to the embodiments shown. It is appreciated that various modifications may occur to those skilled in the art that, while not specifically shown herein, are nevertheless within the true spirit and scope of the invention.

What is claimed is:

1. A method for determining the functional residual lung capacity of a subject, the method comprising:
changing the FiO₂ of a subject's inspirium by a predetermined amount;
for each breath in a series of breaths of said subject subsequent to changing said FiO₂,
determining an expiratory tidal volume measurement value of said breath,
determining an expiratory fractional N₂ tidal volume measurement value of said breath,
multiplying said expiratory tidal volume measurement value of said breath by an absolute difference between said expiratory fractional N₂ tidal volume measurement value of said breath and that of a breath immediately preceding said breath, thereby yielding a first multiplication result,
dividing said first multiplication result by the sum of said absolute differences of each of said breaths, thereby yielding a first division result, and
multiplying said expiratory fractional N₂ tidal volume measurement value of said breath by the sum of said first division results of each of said breaths, thereby yielding a second multiplication result; and
dividing the sum of said second multiplication results of each of said breaths by the absolute difference between said expiratory fractional N₂ tidal volume measurement values of the first and last breaths in said series of breaths, thereby producing a functional residual lung capacity measurement of said subject.

2. A method according to claim 1 wherein said changing step comprises increasing said FiO₂.

3. A method according to claim 1 wherein said changing step comprises decreasing said FiO₂.

4. A method according to claim 1 wherein said changing step comprises changing said FiO₂ by an amount that is within the range of about 20% to about 25% of total inspired volume of said subject.

5. A method according to claim 1 wherein said changing step comprises changing said FiO₂ in accordance with a single step function.

6. A method according to claim 1 and further comprising:
determining a fractional expiratory CO₂ tidal volume of expirium of said subject; and
determining a fractional expiratory O₂ tidal volume of expirium of said subject,
wherein said step of determining said fractional expiratory N₂ tidal volume comprises determining said fractional expiratory N₂ tidal volume as a function of said O₂ and CO₂ fractional expiratory O₂ tidal volumes.

7. A method according to claim 6 wherein said step of determining said fractional expiratory CO₂ tidal volume comprises determining prior to changing said FiO₂.

8. A method according to claim 6 wherein said step of determining said fractional expiratory CO₂ tidal volume comprises determining said fractional expiratory CO₂ tidal volume separately for each of said breath in said series of breaths.

9. A method according to claim 1 wherein said determining steps comprise determining until any of said expiratory fractional tidal volumes reaches a steady state.

10. A method according to claim 9 wherein said determining steps comprise determining until consecutive ones of any of said expiratory fractional tidal volumes differ by less than a predefined amount.

11. A method according to claim 10 wherein said determining steps comprise determining until consecutive ones of any of said expiratory fractional tidal volumes differ by less than <1%.

12. A method according to claim 1 wherein said determining steps comprise determining for predefined number of breaths after any of said expiratory fractional tidal volumes reaches a steady state.

13. A method according to claim 6 wherein said step of determining said fractional expiratory O₂ tidal volume comprises determining using a minimal level of O₂ in said breath after said FiO₂ is increased.

14. A method according to claim 6 wherein said step of determining said fractional expiratory O₂ tidal volume comprises determining using a maximal level of O₂ in said breath after said FiO₂ is decreased.

15. A method according to claim 1 wherein said determining steps comprise associating any of said tidal volumes with any of said breaths where the measurement of said tidal volume is closest in time to the occurrence of said breath after a change in detected in inspirium FiO₂ of said subject.

16. A functional residual lung capacity measurement system, the system comprising:
a ventilation system; and
a functional residual capacity analyzer configured to co-operate with said ventilation system to determine the functional residual lung capacity of a subject that is insufflated with O₂ by said ventilation system, wherein said analyzer is configured to,
a) for each breath in a series of breaths of said subject subsequent to the occurrence of a change in the FiO₂ of a subject's inspirium by a predetermined amount,
determine an expiratory tidal volume measurement value of said breath,
determine an expiratory fractional N₂ tidal volume measurement value of said breath,
multiply said expiratory tidal volume measurement value of said breath by an absolute difference between said expiratory fractional N₂ tidal volume measurement value of said breath and that of a breath immediately preceding said breath, thereby yielding a first multiplication result,
divide said first multiplication result by the sum of said absolute differences of each of said breaths, thereby yielding a first division result, and
multiply said expiratory fractional N₂ tidal volume measurement value of said breath by the sum of said first division results of each of said breaths, thereby yielding a second multiplication result, and
b) divide the sum of said second multiplication results of each of said breaths by the absolute difference between said expiratory fractional N₂ tidal volume measurement values of the first and last breaths in said series of breaths, thereby producing a functional residual lung capacity measurement of said subject.

17. A system according to claim 16 wherein said ventilation system comprises:
an O₂ source;
an O₂ sensor configured to measure inspiratory O₂ between said O₂ source and a subject; and
a flow transducer configured to measure pressure along expiratory and inspiratory channels intermediate said O₂ source and said subject,
wherein said functional residual capacity analyzer is configured to determine any of said tidal volumes using any of said pressure measurement and said inspiratory O₂ measurement.

18. A system according to claim 16 wherein said analyzer is configured to automatically initiate a measurement of said functional residual lung capacity after said change in said FiO₂ occurs.

19. A system according to claim 17 wherein said analyzer is configured to cause said O₂ source to change said FiO₂ of said subject inspirium by said predetermined amount.

20. A system according to claim 19 wherein said O₂ source is configured to change said FiO₂ of said subject inspirium by increasing said FiO₂.

21. A system according to claim 19 wherein said O₂ source is configured to change said FiO₂ of said subject inspirium by decreasing said FiO₂.

22. A system according to claim 19 wherein said O₂ source is configured to change said FiO₂ by an amount that is within the range of about 20% to about 25% of total inspired volume of said subject.

23. A system according to claim 16 wherein said O₂ source is configured to change said FiO₂ in accordance with a single step function.

24. A system according to claim 16 wherein said analyzer is configured to
determine a fractional expiratory CO₂ tidal volume of expirium of said subject,
determine a fractional expiratory O₂ tidal volume of expirium of said subject, and
determine said fractional expiratory N₂ tidal volume as a function of said O₂ and CO₂ fractional expiratory O₂ tidal volumes.

25. A system according to claim 24 wherein said analyzer is configured to determine said fractional expiratory CO₂ tidal volume prior to said change in FiO₂.

26. A system according to claim 24 wherein said analyzer is configured to determine said fractional expiratory CO₂ tidal volume separately for each of said breath in said series of breaths.

27. A system according to claim 16 wherein said analyzer is configured to make any of said determinations until any of said expiratory fractional tidal volumes reaches a steady state.

28. A system according to claim 27 wherein said analyzer is configured to make any of said determinations until consecutive ones of any of said expiratory fractional tidal volumes differ by less than a predefined amount.

29. A system according to claim 28 wherein said analyzer is configured to make any of said determinations until consecutive ones of any of said expiratory fractional tidal volumes differ by less than <1%.

30. A system according to claim 16 wherein said analyzer is configured to make any of said determinations for predefined number of breaths after any of said expiratory fractional tidal volumes reaches a steady state.

31. A system according to claim 24 wherein said analyzer is configured to determine said fractional expiratory O₂ tidal volume using a minimal level of O₂ in said breath after said FiO₂ is increased.

32. A system according to claim 24 wherein said analyzer is configured to determine said fractional expiratory O₂ tidal volume using a maximal level of O₂ in said breath after said FiO₂ is decreased.

33. A system according to claim 16 wherein said analyzer is configured to associate any of said tidal volumes with any of said breaths where the measurement of said tidal volume is closest in time to the occurrence of said breath after a change in detected in inspirium FiO₂ of said subject.

34. A computer program product for determining the functional residual lung capacity of a subject, the computer program product comprising:
a computer readable medium; and
computer program instructions operative to
a) for each breath in a series of breaths of said subject subsequent to the occurrence of a change in the FiO₂ of a subject's inspirium by a predetermined amount,
determine an expiratory tidal volume measurement value of said breath,
determine an expiratory fractional N₂ tidal volume measurement value of said breath,
multiply said expiratory tidal volume measurement value of said breath by an absolute difference between said expiratory fractional N₂ tidal volume measurement value of said breath and that of a breath immediately preceding said breath, thereby yielding a first multiplication result,
divide said first multiplication result by the sum of said absolute differences of each of said breaths, thereby yielding a first division result, and
multiply said expiratory fractional N₂ tidal volume measurement value of said breath by the sum of said first division results of each of said breaths, thereby yielding a second multiplication result, and
b) divide the sum of said second multiplication results of each of said breaths by the absolute difference between said expiratory fractional N₂ tidal volume measurement values of the first and last breaths in said series of breaths, thereby producing a functional residual lung capacity measurement of said subject,
wherein said program instructions are stored on said computer readable medium.
